(19) United States
(12) Patent Application Publication
(10) Pub. No.: US 2016/0304586 A1
(43) Pub. Date: Oct. 20, 2016

(54) PROCESS FOR PREPARING INFLUENZA VACCINES

(71) Applicant: CRUCELL HOLLAND B.V., Leiden (NL)

(72) Inventors: Maria Anna Rosalie PAGANY, Almere (NL); Eufemia G.M. WALRAVEN-BERKHOFF, Nootdorp (NL); Martinus J.M. VAN OOIJ, Alphen aan den Rijn (NL)

(73) Assignee: Crucell Holland B.V., Leiden (NL)

(21) Appl. No.: 15/100,930

(22) PCT Filed: Dec. 4, 2014
(86) PCT No.: PCT/EP2014/076548

§ 371 Date: Jun. 1, 2016

(30) Foreign Application Priority Data
Dec. 5, 2013 (EP) 13195833.2

(51) Int. Cl. C07K 16/10; G01N 33/577; G01N 33/569; A61K 39/145; C12N 7/00

(52) U.S. Cl. CPC C07K 16/1018; A61K 39/145; C12N 7/00; G01N 33/56983; G01N 33/577; C07K 2317/565; C07K 2317/76; C12N 2760/16134; C12N 2760/16234; C12N 2760/16163; C12N 2760/16263; G01N 2333/11

(57) ABSTRACT

This disclosure provides a novel process for testing an influenza virus preparation for the presence of extraneous agents. The disclosure further provides a process for preparing an influenza vaccine.

### CROSS-REFERENCE TO RELATED APPLICATIONS

This application is a national phase entry under 35 U.S.C. §371 of International Patent Application PCT/EP2014/076548, filed Dec. 4, 2014, designating the United States of America and published in English as International Patent Publication WO 2015/082600 A1 on Jun. 11, 2015, which claims the benefit under Article 8 of the Patent Cooperation Treaty to European Patent Application Serial No. 13195833.2, filed Dec. 5, 2013.

### TECHNICAL FIELD

This application relates generally to medicine and more particularly to the production and quality control of influenza virus vaccines, such as influenza virus vaccines derived from influenza viruses that have been grown in a culture of a mammalian cell line.

### BACKGROUND

Influenza viruses for use in human influenza vaccines traditionally are grown on embryonated hen eggs. Continuous cell lines, such as, e.g., PER.C6® cells (human fetal retina), Madin Darby canine kidney (MDCK) cells or Vero (African green monkey kidney) cells provide alternatives to embryonated hen eggs for use as substrates. In contrast to the use of embryonated eggs, cell-based manufacturing allows for a better controlled substrate and a closed production process utilizing bioreactors. Egg-based influenza vaccine production is dependent on the availability of embryonated eggs, which is at risk in the event of outbreaks of bird diseases. In addition, the use of a continuous cell line will allow manufacturers to respond more quickly to the emergence of new strains of influenza in a pandemic situation.

The use of mammalian cell substrates for influenza vaccine production involves culturing the cells under conditions that are optimized for viral growth and replication. It is known that (viral) pathogens other than influenza virus may also grow in the cell culture under such conditions, thereby leading to potential contamination of the final vaccine product. Manufacturing of viral vaccines, therefore, is governed by strict rules and regulations, also with respect to the demonstration of the absence of extraneous or adventitious agents. Since the seasonal influenza vaccine is a viral vaccine, testing for extraneous viral adventitious agents is, in particular, required.

Compendial test methods are in place to determine if extraneous viral contaminants are present in a product sample. To perform such tests, prior specific neutralization of the influenza virus is required to determine cytopathic effects, hemadsorption and/or hemagglutination activities of extraneous viruses. After specific neutralization of the influenza virus, the virus composition is added to selected cell lines for in vitro testing and/or is tested in in vivo animal models. If the cell lines exhibit pathogenic effects, or the animals show evidence of infection attributable to the virus composition, this indicates that the composition contains extraneous or adventitious agents. For neutralizing the influenza viruses in the compositions to be tested, specific antisera are generated directed against the candidate influenza vaccine strains, preferably in species different from the species from which the primary influenza virus was isolated, e.g., sheep. Generating specific, adventitious virus-free, neutralizing antisera with sufficient capacity to avoid a breakthrough of high titer influenza viruses during testing is regarded as the most critical issue for successful virus seed testing. It is a time-consuming process, which has to be done almost each year since circulating influenza viruses in humans are subject to permanent antigenic drift, which requires annual adaptation of the influenza vaccine formulation to ensure the closest possible match between the influenza vaccine strains and the circulating influenza strains.

In view of the severity of the respiratory illness caused by influenza A and influenza B viruses, as well as the high economic impact of the seasonal epidemics and the continuing risk for pandemics, there is an ongoing need for improved methods for preparing safe and effective influenza vaccines, in particular, methods for preparing safe and effective influenza vaccines grown on cell culture.

### BRIEF SUMMARY

This disclosure provides a novel method for testing an influenza virus composition for the presence of extraneous agents, wherein the influenza virus is neutralized prior to extraneous agent testing using one or more unique influenza virus-neutralizing monoclonal antibodies. According to the disclosure, use is made of unique monoclonal antibodies directed against a broad range of influenza viruses. These universal antibodies potently neutralize a wide spectrum (over 50 years of evolution) of different subtypes and strains of influenza A virus and influenza B strains of both the Yamagata and Victoria lineage. After specific neutralization of the influenza virus, the influenza virus composition, e.g., the influenza virus seed or virus harvest, can be tested for the presence of extraneous agents using compendial assays and techniques well known in the art.

This disclosure also provides a method for preparing an influenza vaccine, comprising a step of testing an influenza virus composition for the presence of extraneous agents, wherein the influenza virus is neutralized prior to extraneous agent testing using one or more of the universal influenza virus-neutralizing monoclonal antibodies.

This disclosure further provides the use of an influenza virus-neutralizing monoclonal antibody, selected from the group consisting of CR6323, CR8043 and CR11039, and/or functional variants and/or antigen-binding fragments thereof, as a neutralizing agent for influenza virus extraneous agent testing.

In the field of pharmaceutical industry, there is the demand to produce compositions that are free of contaminants such as extraneous (or adventitious) agents, as these may jeopardize the safety of the product. This demand is particularly challenging in the viral vaccine field. Manufacturers of human viral vaccines have to ensure that their products are free from extraneous agents. Therefore, regulatory agencies require vaccine manufacturers to evaluate the safety of a vaccine based on a standard battery of safety tests. Within the meaning of this disclosure, the term “extraneous agent” or “adventitious agent” relates to a contaminating agent, e.g., microorganism, that has been unintentionally introduced into the manufacturing process of a biological product and, thus, may be present in the composition to be tested. Thus, an “adventitious” or “extraneous” agent is an agent that is not intended to be included in the composition and that can adversely influence the properties of a product containing the composition, such as, e.g., an infectious agent (pathogen), i.e., an agent capable of infecting a human or animal. Such an infectious agent can be a microorganism, e.g., bacteria, fungi, mycoplasma/spiroplasma, mycobacteria, rickettsia, viruses, protozoa, parasites, TSE agent, and/or parts thereof. The infectious agents are also often able to grow in systems such as cell cultures that are used for the production of the biological product.

This disclosure, in particular, relates to the quality control of influenza vaccines. Numerous protocols are in place to determine if extraneous agents are present in an influenza vaccine composition. In particular, influenza virus compositions, such as virus seed lots (Working Virus Seed: WVS) and/or virus harvest preparations, need to be tested according to regulations and recommendations as, e.g., laid down in Pharm. Eur. 2.6.16: “Tests for extraneous agents in viral vaccines for human use”; 9CFR113.55: “Detection of extraneous agents” in Master Seed Virus and U.S. FDA Guidance for Industry (GFI): “Characterization and Qualification of Cell Substrate and Other Biological materials Used in the Production of Viral Vaccines for Infectious Disease.” For these tests, the influenza viruses need to be specifically neutralized prior to inoculation into cell culture and animals to circumvent the generation of false positive results, such as viral cytopathic effects, hemadsorption and/or hemagglutination activities of the influenza viruses themselves.

This disclosure provides a novel method for testing for the presence of extraneous agent(s) in a composition comprising influenza virus, comprising the steps of neutralizing the influenza virus in the composition using one or more influenza virus-neutralizing monoclonal antibodies, and/or antigen-binding fragments thereof. As outlined above, according to the disclosure, use is made of specific unique monoclonal antibodies directed against a broad range of influenza viruses. These universal recombinant monoclonal antibodies potently neutralize a wide spectrum (over 50 years of evolution) of different subtypes and strains of influenza A virus and influenza B strains of both the Yamagata and Victoria lineage.

This disclosure also provides a method for preparing an influenza vaccine, comprising a step of testing an influenza virus composition for the presence of extraneous agents, wherein the influenza virus in the composition is neutralized prior to testing, using one or more influenza virus-neutralizing monoclonal antibodies, and/or antigen-binding fragments thereof.

In certain embodiments, the methods further comprise a step of determining whether the composition comprises extraneous agents in vitro, in vivo and/or in ovo. By applying the methods according to the disclosure, the presence of extraneous or adventitious agents in a composition comprising an influenza virus can be tested in an efficient, expeditious, and reliable manner.

In certain embodiments, the influenza virus-neutralizing antibodies are human monoclonal antibodies. In certain embodiments, the influenza virus-neutralizing antibodies are recombinant monoclonal antibodies.

In certain embodiments, the influenza virus has been propagated in a cell culture, preferably a mammalian cell culture, such as a human or simian cell culture. The influenza virus composition to be tested can be derived from the cell culture at any stage, which means that the influenza virus composition can either be a precursor of the final product or the final product itself, or any product in between. Preferably, the influenza virus composition to be tested is an influenza seed virus (also referred to as “Working Virus Seed,” WVS). An influenza seed virus within the meaning of this disclosure is an influenza virus that is intended to be used for the production of a vaccine.

In certain embodiments, the one or more influenza virus-neutralizing antibodies are selected from the group consisting of CR6323, CR8043, and CR11039, and/or functional variants and/or antigen-binding fragments thereof. The specific antibody or antibodies to be used will depend on the type of influenza virus to be neutralized. Recently, human monoclonal antibodies capable of broadly neutralizing influenza A viruses of phylogenetic group 1 (e.g., CR6323 and functional variants thereof, as described in WO 2008/028946) or influenza viruses of phylogenetic group 2 (e.g., CR8043 and functional variants thereof, as described in WO 2010/130636), or influenza B viruses (e.g., CR11039 and functional variants thereof, as described in the co-pending patent application EP12158525.1) have been identified. In the research that led to the disclosure, the specific neutralization of seasonal influenza vaccine virus seeds was examined using these unique recombinant monoclonal antibodies. These universal monoclonal antibodies potently neutralize a wide spectrum of different subtype and strains of influenza A virus and influenza B strains of both the Yamagata and Victoria lineage, as described by Ekiert et al. (Science 338:843-850, 2011; Science 324:246-251, 1009) and Dreyfus et al. (Science 337:1343-1348, 2012). Thus, prior studies have shown that CR8043 potently neutralizes a wide spectrum (i.e., over 50 years of virus evolution) of H3 influenza strains as well as H7 and H10 viruses (all belonging to group 2 influenza viruses). CR6323 has been shown to potently neutralize H1, H5, H9, and some H2 (all belonging to group 1 influenza viruses), but does not bind to group 2 influenza viruses. CR11039 has been shown to potently neutralize a wide spectrum of influenza B strains of both the Yamagata and Victoria lineage.

According to this disclosure, it has surprisingly been found that use can be made of these unique specific monoclonal antibodies, functional variants and/or antigen-binding fragments thereof, for specifically neutralizing the influenza virus strains, thus obviating the need for generating specific polyclonal antisera against the different influenza virus strains used for preparing the influenza vaccines. Thus, it was found that these monoclonal antibodies effectively neutralized seasonal influenza vaccine seeds in vitro, in ovo, and in vivo, during testing for extraneous agents. Therefore, these monoclonal antibodies present promising candidates as neutralizing agents for extraneous agents testing in the manufacture of influenza vaccines. The amino acid sequences of the heavy and light chain complementarity-determining regions (CDRs) and variable regions of the antibodies to be used are given below.

In certain embodiments, the one or more influenza virus-neutralizing antibodies are selected from: (i) an antibody comprising a heavy chain CDR1 region comprising SEQ ID NO:1, a heavy chain CDR2 region comprising SEQ ID NO:2 and a heavy chain CDR3 region comprising SEQ ID NO:3; and a light chain CDR1 region comprising SEQ ID NO:4, a light chain CDR2 region comprising SEQ ID NO:5 and a light chain CDR3 region comprising SEQ ID NO:6; (ii) an antibody comprising a heavy chain CDR1 region comprising SEQ ID NO:7, a heavy chain CDR2 region comprising SEQ ID NO:8 and a heavy chain CDR3 region comprising SEQ ID NO:9; and a light chain CDR1 region comprising SEQ ID NO:10, a light chain CDR2 region comprising SEQ ID NO:11 and a light chain CDR3 region comprising SEQ ID NO:12; and (iii) an antibody comprising a heavy chain CDR1 region comprising SEQ ID NO:13, a heavy chain CDR2 region comprising SEQ ID NO:14 and a heavy chain CDR3 region comprising SEQ ID NO:15; and a light chain CDR1 region comprising SEQ ID NO:16, a light chain CDR2 region comprising SEQ ID NO:17 and a light chain CDR3 region comprising SEQ ID NO:18.

In certain embodiments, the influenza virus-neutralizing antibodies are selected from: (i) an antibody comprising a heavy chain variable region comprising the amino acids 1-120 of SEQ ID NO:20 and a light chain variable region comprising the amino acid residues 1-109 of SEQ ID NO:22; (ii) an antibody comprising a heavy chain variable region comprising the amino acid sequence of SEQ ID NO:24 and a light chain variable region comprising the amino acid sequence of SEQ ID NO:26; and (iii) an antibody comprising a heavy chain variable region comprising the amino acid sequence of SEQ ID NO:28 and a light chain variable region comprising the amino acid sequence of SEQ ID NO:30.

According to the disclosure, use is made of substantially pure preparations of monoclonal antibodies for neutralization of influenza virus, i.e., antibody preparations comprising only the one or more recombinant influenza virus-neutralizing antibodies, in known and set concentrations, which can be used for neutralizing a wide spectrum of influenza virus strains. In contrast, the traditionally used antisera comprise several polyclonal antibodies in variable concentrations. Due to possible cytopathic effect of serum, antisera need to be diluted prior to use for neutralization of influenza virus, which may result in a lack of neutralization due to low concentrations of the specific neutralizing antibodies.

Use can be made of the recombinant monoclonal antibodies, and/or of functional variants and/or antigen-binding fragments thereof as a neutralizing agent for extraneous agent testing. As used herein, “functional variants” are antibodies with heavy and/or light chain variable regions comprising an amino acid sequence that is at least 95%, preferably at least 97%, for instance, at least 98% or 99% homologous or identical to the amino acid sequences of the heavy and/or light chain variable regions of CR6323, CR8043 or CR11039, as defined above, and are capable of competing for binding to hemagglutinin with the “parent” antibodies CR6323, CR8043 and CR11039, respectively, and having influenza virus-neutralizing activity. In preferred embodiments, a functional variant comprises heavy and/or light chain variable sequences that are at least 95%, 97%, 98%, or 99% identical in amino acid sequence with the parent antibody. The term “functional variant,” as used herein, thus refers to a monoclonal antibody that comprises a heavy and/or light chain variable region having an amino acid sequence that is altered by one or more amino acids compared to the amino acid sequences of the parental monoclonal antibody. The functional variant may have conservative sequence modifications including amino acid substitutions, additions and deletions. Amino acid modifications can be introduced by standard techniques known in the art, such as site-directed mutagenesis, molecular cloning, oligonucleotide-directed mutagenesis and random PCR-mediated mutagenesis in the nucleic acid encoding the antibodies. Conservative amino acid substitutions include the ones in which the amino acid residue is replaced with an amino acid residue having similar structural or chemical properties (silent mutations). Families of amino acid residues having similar side chains have been defined in the art. Furthermore, a functional variant may have non-conservative amino acid substitutions, e.g., replacement of an amino acid with an amino acid residue having different structural or chemical properties (non-silent mutations). Similar minor variations may also include amino acid deletions or insertions, or both. Guidance in determining which amino acid residues may be substituted, inserted, or deleted without abolishing immunological activity may be found using computer programs well known in the art. Computer algorithms such as inter alia Gap or Bestfit known to a person skilled in the art can be used to optimally align amino acid sequences to be compared and to define similar or identical amino acid residues.

Functional variants of CR6323 are, for instance, described in WO 2008/028946. Functional variants of CR8043 are, for instance, described in WO 2010/130636. Functional variants of CR11039 are, for instance, described in co-pending application EP12158525.1.

As used herein, an “antigen-binding fragment” according to this disclosure, refers to a peptide or polypeptide comprising an amino acid sequence of at least 5, 10, 15, 20, 25, 30, 35, 40, 50, 60, 70, 80, 90, 100, 125, 150, 175, 200, or 250 contiguous amino acid residues of the amino acid sequence of an antibody of the disclosure, wherein the fragment competes with the intact antibody for specific binding to its binding partner, i.e., hemagglutinin (HA). Regardless of structure, the antigen-binding fragment thus binds to the same antigen that is recognized by the intact immunoglobulin. Antigen-binding fragments include, inter alia, Fab, F(ab′), F(ab′)2, Fv, dAb, Fd, complementarity-determining region (CDR) fragments, single-chain antibodies (scFv), bivalent single-chain antibodies, single-chain phage antibodies, diabodies, triabodies, tetrabodies, and (poly)peptides that contain at least a fragment of an immunoglobulin that is sufficient to confer specific antigen binding to the (poly)peptide, etc. The above fragments may be produced synthetically or by enzymatic or chemical cleavage of intact immunoglobulins or they may be genetically engineered by recombinant DNA techniques. The methods of production are well known in the art and are described, for example, in Antibodies: A Laboratory Manual, edited by E. Harlow and D. Lane (1988), Cold Spring Harbor Laboratory, Cold Spring Harbor, N.Y.

The methods of the disclosure are particularly suitable for in vitro assays for the detection of adventitious viruses. In certain embodiments, cells that are used for such in vitro assays are selected from PER.C6® cells, MDCK cells, Simian cells, like Vero cells, and human diploid cells, like MRC cells.

The methods of the disclosure are, in particular, suitable for testing and/or preparing an influenza vaccine from influenza virus that has been grown in a culture of a mammalian cell line. The risk of extraneous agents is regarded to be higher in cell-produced influenza viruses than in egg-based influenza viruses because of the susceptible nature of the cells. In certain embodiments, the mammalian cell line is a PER.C6® cell line, a MDCK cell line, or a Vero cell line. Examples of infectious viruses and bacteria that might infect such cells, and thus representing potential extraneous or adventitious agents to be tested according to the disclosure include, for example, viruses selected from the group consisting of Pneumovirinae, such as the Pneumovirus genus, including respiratory syncytial virus (RSV); Morbilliviruses of the Paramyxoviridae family, such as measles virus; Enteroviruses of the Picornaviridae family, such as Coxsackie viruses, for instance, coxsackie B5, echo viruses, enteroviruses group A-D, and Rhinoviruses of the Picornaviridae family, such as M-strains of Rhino virus; mammalian Reoviridae, in particular, orthoreoviruses (e.g., mammalian reoviruses such as reovirus 1, 2, and 3), avian Reoviridae, in particular, orthoreoviruses, such as avian reoviruses; rotaviruses; members of the Retroviridae, for instance, the Orthoretrovirinae, such as HIV-1, HIV-2, SIV; Metapneumoviruses of the Paramyxoviridae family, such as human metapneumovirus (HMPV), or Parainfluenza virus (PIV) type 1, 2, 3, and 4; Rubulaviruses of the Paramyxoviridae family, such as mumps virus; Togaviridae, such as Rubellavirus; Coronaviridae, such as the SARS coronavirus and other human coronaviruses such as coronavirus OC43, 229E, NL63, and HKU1; Polyomaviridae, such as the SV-40 polyomavirus, the BK polyomavirus and the JC polyomavirus; Porcine circoviruses; Porcine picornaviruses, such as swine vesicular disease virus (SVDV) and Teschen-Talfan virus; members of the Parvoviridae, such as canine parvovirus (CPV), bocaviruses or porcine parvoviruses; members of the Orthomyxoviridae, including influenza virus type A and B; the Herpesviridae, such as herpes simplex virus 1 and 2, human herpes simplex virus type 6, 7 or 8, cytomegalovirus and Epstein Barr virus and Varicella Zoster virus (VZV), also known as human herpes virus 3 (HHV-3); the Adenoviridae, such as the adenoviruses, including human, simian and avian adenovirus, such as avian adenovirus 1; avian circoviruses; members of the Papillomaviridae, including human papilloma virus; members of the Flaviviridae, such as the West Nile virus; and Birnaviridae, such as infectious bursal disease virus (also known as gumboro virus); and bacteria, such as Chlamydia bacteria, including C. trachomatis, C. pneumoniae and C. psittaci; Mycobacteria, and Mycoplasma.

The influenza virus compositions to be tested may be used for the preparation of both seasonal and pandemic influenza vaccines. In addition, the influenza virus compositions to be tested may comprise both human and non-human (e.g., avian) influenza viruses. The influenza virus composition to be tested may be derived from any stage in the manufacturing process of influenza virus vaccines.

There are three known types of influenza virus (types A, B and C). The type A and type B viruses are the agents responsible for the influenza seasonal epidemics and type A viruses are also responsible for pandemics observed in humans. Influenza A viruses generally are classified into influenza virus subtypes based on variations in antigenic regions of hemagglutinin (HA) and neuraminidase (NA). These viral surface glycoproteins are required for viral attachment and cellular release. Currently, sixteen subtypes of HA (H1-H16) and nine NA (N1-N9) antigenic variants are known in influenza A viruses. Influenza virus subtypes are also classified by reference to their phylogenetic group: inter alia the human influenza virus H1, H2, H5 and H9 subtypes in phylogenetic group 1 and inter alia the human influenza virus H3, H4 and H7 subtypes in phylogenetic group 2. The antigenic variations in HA within the influenza type B virus strains are less pronounced than those observed within the type A strains. However, two genetically and antigenically distinct lineages of influenza B virus are circulating in humans, as represented by the B/Yamagata/16/88 (also referred to as B/Yamagata) and B/Victoria/2/87 (B/Victoria) lineages.

According to the disclosure, the influenza virus to be tested can be any influenza A or B virus strain(s). The method of the disclosure is applicable to both seasonal and pandemic vaccines. In certain embodiments of this disclosure, the influenza virus is an influenza A virus strain derived from phylogenetic group 1, e.g., an influenza virus strain comprising HA of the H1 (such as an H1N1 influenza virus), H2 (such as an H2N2 influenza virus), an H5 (such as an H5N1 influenza virus strain) or H9 subtype (such as an H9N2 influenza virus). In other embodiments, the influenza virus is an influenza A virus strain derived from phylogenetic group 2, e.g., an influenza virus comprising HA of the H3 (such as an H3N2 influenza virus), H7 (such as an H7N7 influenza virus), or H10 subtype. In further embodiments, the influenza virus is an influenza B virus strain from the B/Yamagata or B/Victoria lineage. The methods of the disclosure are, in particular, useful in cases where it is very difficult to raise neutralizing antisera.

This disclosure further relates to the use of an influenza virus-neutralizing recombinant monoclonal antibody, selected from the group consisting of CR6323, CR8043 and CR11039, and/or functional variants and/or antigen-binding fragments thereof, as a neutralizing agent for influenza virus extraneous agent testing.

The disclosure also relates to an influenza vaccine obtained or obtainable by the process as described herein. The vaccine may be an inactivated, a live attenuated or cold-adapted influenza vaccine. In certain embodiments, the vaccine is an inactivated virus vaccine. Virus inactivation typically involves treatment with a chemical such as formalin or [beta]-propiolactone. Where an inactivated virus is used, the vaccine may be a whole virus, a split virus, viral subunits or a virosomal influenza vaccine. Split viruses are obtained by treating virions with detergents e.g., ethyl ether, polysorbate 80, deoxycholate, tri-N-butyl phosphate, TRITON X®-100, Triton N101, cetyltrimethylammonium bromide, etc.) to produce subvirion preparations. Subunit vaccines comprise the influenza surface antigens hemagglutinin and neuraminidase.

The vaccines of the disclosure typically include antigens from at least one strain of influenza A virus and/or at least one strain of influenza B virus. In certain embodiments, the vaccines typically include two influenza A strains (e.g., H1N1 and H3N2) and one or two influenza B strains. Trivalent or quadrivalent influenza vaccines are preferred. The disclosure is also suitable for testing and/or preparing viruses from pandemic strains, such as H5 or H7 strains, i.e., strains to which the human population is immunologically naive. Vaccines in pandemic situations may be monovalent, or they may be based on a normal trivalent vaccine supplemented by a pandemic strain.

Administration of the vaccine according to the disclosure can be performed using standard routes of administration. Non-limiting examples include parenteral administration, such as intravenous, intradermal, transdermal, intramuscular, subcutaneous, etc., or mucosal administration, e.g., intranasal, oral, and the like. The vaccines may be administered more than one time, i.e., in a so-called homologous prime-boost regimen. In certain embodiments, the vaccine is for administration to a patient by injection (e.g., subcutaneous injection or intramuscular injection), by an intranasal route, by an intradermal route, by a transcutaneous route, or by a transdermal route.

Vaccines prepared according to the disclosure may be used to treat both children and adults. Thus, prevention and/or treatment may be targeted at patient groups that are susceptible to infection with influenza virus. Such patient groups include, but are not limited to, e.g., the elderly (e.g., ≧50 years old, ≧60 years old, and preferably ≧65 years old), the young (e.g., ≦5 years old, ≦1 year old), hospitalized patients and patients who have been treated with an antiviral compound but have shown an inadequate antiviral response. Influenza vaccines are currently recommended for use in pediatric and adult immunization, from the age of 6 months.

In certain embodiments, the vaccine further comprises an adjuvant. The adjuvant may be administered before, concomitantly with, or after administration of the vaccine. Examples of suitable adjuvants include saponin formulations, such as, for example, QS21 and immunostimulating complexes (ISCOMS) (see, e.g., U.S. Pat. No. 5,057,540; WO 90/03184, WO 96/11711, WO 2004/004762, WO 2005/002620); bacterial or microbial derivatives, examples of which are monophosphoryl lipid A (MPL), 3-O-deacylated MPL (3dMPL), CpG-motif containing oligonucleotides, ADP-ribosylating bacterial toxins or mutants thereof, such as E. coli heat labile enterotoxin LT, cholera toxin CT, pertussis toxin PT, or tetanus toxoid TT, and Matrix M (Isconova). However, since virosomes may have adjuvanting activity, when the influenza vaccine is a virosomal influenza, no additional adjuvants may be needed.

The disclosure is further illustrated in the following Examples.

### DETAILED DESCRIPTION

### Examples

### Example 1

### Use of Influenza Monoclonal Antibodies as a Neutralizing Agent for Influenza Vaccine Extraneous Agents Testing

In this Example, suitable conditions were tested and evaluated for a cell-based in vitro adventitious virus assay for testing of influenza virus seed stocks neutralized by the unique monoclonal antibodies CR8043, CR6325 and CR11039. The influenza viruses were grown in mammalian cells, in particular, PER.C6® cells. Feasibility experiments were performed prior to those performed during definite AVA (adventitious viruses analysis) testing to comply with Ph.Eur 2.6.16. The results were evaluated based on viral cytopathic effects (CPE) and hemagglutination activity (HA).

Results were obtained from at least two independent repeated experiments for each influenza virus subtype and corresponding monoclonal antibody. Neutralization of individual influenza virus seed subtypes was assessed in eight separate experiments. In experiments 1, 2, 3, and 8, neutralization of influenza virus A/H3N2 with CR8043 was assessed. In experiments 4 and 7, neutralization of influenza virus A/H1N1 with CR6323 was assessed. In experiments 5 and 6, neutralization of influenza B virus with CR11039 was assessed. In each experiment, a range of dilutions was tested in duplicate against a range of concentrations of the influenza antibodies. Both CPE and HA were scored. Since the lower limits of detection between both parameters differ, i.e., HA is more readily detected than CPE, the observed scores for CPE and HA were not identical. For the determination of the neutralizing capacity of each influenza antibody, the lowest limit of detection was considered.

The results are summarized in the following Tables:

TABLE 1
Experiment 1 - influenza virus A/H3N2 and CR8043
Schematic overview MW96: CPE

| | | | | Undiluted | | | | | | | | | | Non- | |
|---|---|---|---|---|---|---|---|---|---|---|---|---|---|---|---|
| | | | | sample | | 1 | | 0.1 | | 0.01 | | 0.001 | | infected | |
| MOI | | | | 1 | 2 | 3 | 4 | 5 | 6 | 7 | 8 | 9 | 10 | 11 | 12 |
| Conc. | 40 | μg/mL | A | + | + | O | O | O | O | O | + | ? | ? | ? | ? |
| FluMabs | 20 | μg/mL | B | + | + | O | O | O | O | O | O | O | O | O | ? |
| | 10 | μg/mL | C | + | + | O | O | O | O | O | O | O | O | O | ? |
| | 5 | μg/mL | D | + | + | O | O | O | O | O | O | O | O | O | O |
| | 2.5 | μg/mL | E | + | + | + | + | O/+ | O | O | O | O | O | O | O |
| | 1.25 | μg/mL | F | + | + | + | + | + | + | + | + | O | O | O | O |
| | 0.625 | μg/mL | G | + | + | + | + | + | + | + | + | + | + | O | O |

| | | | | | | | | | | | | | |
|---|---|---|---|---|---|---|---|---|---|---|---|---|---|
| No FluMAbs | H | + | + | + | + | + | + | + | + | + | + | O | O |

+: CPE
O: No CPE
?: Due to evaporation, cells could not be scored
O/+: No consensus between scores

TABLE 2
Experiment 1 - influenza virus A/H3N2 and CR8043
Schematic overview MW96: HA

| | | | | Undiluted | | | | | | | | | | Non- | |
|---|---|---|---|---|---|---|---|---|---|---|---|---|---|---|---|
| | | | | sample | | 1 | | 0.1 | | 0.01 | | 0.001 | | infected | |
| MOI | | | | 1 | 2 | 3 | 4 | 5 | 6 | 7 | 8 | 9 | 10 | 11 | 12 |
| Conc. | 40 | μg/mL | A | + | + | + | + | + | O | O | ? | O | O | O | O |
| FluMabs | 20 | μg/mL | B | + | + | + | + | O | O | O | O | O | O | ? | ? |
| | 10 | μg/mL | C | + | + | + | + | O | O | O | O | O | O | O | ? |
| | 5 | μg/mL | D | + | + | + | + | O | O | O | O | O | O | O | O |
| | 2.5 | μg/mL | E | + | + | + | + | + | + | + | + | + | O | O | O |
| | 1.25 | μg/mL | F | + | + | + | + | + | + | + | + | O | + | O | O |
| | 0.625 | μg/mL | G | + | + | + | + | + | + | + | + | + | + | O | O |

| | | | | | | | | | | | | | |
|---|---|---|---|---|---|---|---|---|---|---|---|---|---|
| No FluMAbs | H | + | + | + | + | + | + | + | + | + | + | O | ? |

+: HA
O: No HA
?: Due to evaporation, cells could not be scored

TABLE 3
Experiment 2 - influenza virus A/H3N2 and FluMAbs CR8043
Schematic overview MW96: CPE

| | | | | Undiluted | | | | | | | | | | Non- | |
|---|---|---|---|---|---|---|---|---|---|---|---|---|---|---|---|
| | | | | sample | | 1 | | 0.1 | | 0.01 | | 0.001 | | infected | |
| MOI | | | | 1 | 2 | 3 | 4 | 5 | 6 | 7 | 8 | 9 | 10 | 11 | 12 |
| Conc. | 320 | μg/mL | A | + | + | O | O | + | O | O | O | O | O | O | O |
| FluMabs | 160 | μg/mL | B | + | + | O | O | O | O | O | O | O | O | O | O |
| | 80 | μg/mL | C | + | + | O | O | O | + | O | O | O | O | O | O |
| | 40 | μg/mL | D | + | + | O | O | O | O | O | O | O | O | O | O |
| | 20 | μg/mL | E | + | + | O | O | O | O | O | O | O | O | O | O |
| | 10 | μg/mL | F | + | + | O | O | O | O | O | O | O | O | O | O |
| | 5 | μg/mL | G | + | + | + | + | + | O | O | O | O | O | O | O |

| | | | | | | | | | | | | | |
|---|---|---|---|---|---|---|---|---|---|---|---|---|---|
| No FluMAbs | H | + | + | + | + | + | + | + | + | + | + | O | O |

+: CPE
O: No CPE

TABLE 4
Experiment 2 - influenza virus A/H3N2 and FluMAbs CR8043
Schematic overview MW96: HA

| | | | | Undiluted | | | | | | | | | | Non- | |
|---|---|---|---|---|---|---|---|---|---|---|---|---|---|---|---|
| | | | | sample | | 1 | | 0.1 | | 0.01 | | 0.001 | | infected | |
| MOI | | | | 1 | 2 | 3 | 4 | 5 | 6 | 7 | 8 | 9 | 10 | 11 | 12 |
| Conc. | 320 | μg/mL | A | + | + | + | + | + | O | O | O | O | O | O | O |
| FluMabs | 160 | μg/mL | B | + | + | + | + | O | O | O | O | O | O | O | O |
| | 80 | μg/mL | C | + | + | + | + | + | + | O | O | O | O | O | O |
| | 40 | μg/mL | D | + | + | + | + | + | + | O | O | O | O | O | O |
| | 20 | μg/mL | E | + | + | + | + | + | + | O | O | O | O | O | O |
| | 10 | μg/mL | F | + | + | + | + | + | + | O | O | O | O | O | O |
| | 5 | μg/mL | G | + | + | + | + | + | + | + | + | O | O/+ | O | O |

| | | | | | | | | | | | | | |
|---|---|---|---|---|---|---|---|---|---|---|---|---|---|
| No FluMAbs | H | + | + | + | + | + | + | + | + | + | + | O | O |

+: HA
O: No HA
?: Due to evaporation, cells could not be scored

TABLE 5
Experiment 3 - influenza virus A/H3N2 and FluMAbs CR8043
Schematic overview MW96: CPE

| | | | | Undiluted | | | | | | | | | | Non- | |
|---|---|---|---|---|---|---|---|---|---|---|---|---|---|---|---|
| | | | | sample | | 1 | | 0.1 | | 0.01 | | 0.001 | | infected | |
| MOI | | | | 1 | 2 | 3 | 4 | 5 | 6 | 7 | 8 | 9 | 10 | 11 | 12 |
| Conc. | 320 | μg/mL | A | ? | ? | O | O | O | + | O | O | O | O | O | O |
| FluMabs | 160 | μg/mL | B | ? | ? | O | O | O | O | O | O | O | O | O | O |
| | 80 | μg/mL | C | ? | ? | O | O | O | + | O | O | O | O | O | O |
| | 40 | μg/mL | D | ? | ? | O | O | + | O | O | O | O | O | O | O |
| | 20 | μg/mL | E | ? | ? | O | O | O | O | O | O | O | O | O | O |
| | 10 | μg/mL | F | ? | ? | O | O | O | O | O | O | O | O | O | O |
| | 5 | μg/mL | G | + | + | + | + | O | O | O | O | O | O | O | O |

| | | | | | | | | | | | | | |
|---|---|---|---|---|---|---|---|---|---|---|---|---|---|
| No FluMAbs | H | + | + | + | + | + | + | + | + | + | + | O | O |

+: CPE
O: No CPE

TABLE 6
Experiment 3 - influenza virus A/H3N2 and FluMAbs CR8043
Schematic overview MW96: HA

| | | | | Undiluted | | | | | | | | | | Non- | |
|---|---|---|---|---|---|---|---|---|---|---|---|---|---|---|---|
| | | | | sample | | 1 | | 0.1 | | 0.01 | | 0.001 | | infected | |
| MOI | | | | 1 | 2 | 3 | 4 | 5 | 6 | 7 | 8 | 9 | 10 | 11 | 12 |
| Conc. | 320 | μg/mL | A | + | + | + | + | O | O | O | O | O | O | O | O |
| FluMabs | 160 | μg/mL | B | + | + | + | + | O | O | O | O | O | O | O | O |
| | 80 | μg/mL | C | + | + | + | + | O | O/+ | O | O | O | O | O | O |
| | 40 | μg/mL | D | + | + | + | + | + | O | O | O | O | O | O | O |
| | 20 | μg/mL | E | + | + | + | + | + | + | O | O | O | O | O | O |
| | 10 | μg/mL | F | + | + | + | + | + | + | + | + | O | O | O | O |
| | 5 | μg/mL | G | + | + | + | + | + | + | + | + | + | O | O | O |

| | | | | | | | | | | | | | |
|---|---|---|---|---|---|---|---|---|---|---|---|---|---|
| No FluMAbs | H | + | + | + | + | + | + | + | + | + | + | O | O |

+: HA
O: No HA
O/+: No consensus between scores

TABLE 7
Experiment 4 - influenza virus A/H1N1 and FluMAbs CR6323
Schematic overview MW96: CPE

| | | | | Undiluted | | | | | | | | | | Non- | |
|---|---|---|---|---|---|---|---|---|---|---|---|---|---|---|---|
| | | | | sample | | 1 | | 0.1 | | 0.01 | | 0.001 | | infected | |
| MOI | | | | 1 | 2 | 3 | 4 | 5 | 6 | 7 | 8 | 9 | 10 | 11 | 12 |
| Conc. | 320 | μg/mL | A | + | + | O | O | O | O | O | O | O | O | O | O |
| FluMabs | 160 | μg/mL | B | + | + | O | O | O | O | O | O | O | O | O | O |
| | 80 | μg/mL | C | + | + | O | O | O | O | O | O | O | O | O | O |
| | 40 | μg/mL | D | + | + | O | O | O | O | O | O | O | O | O | O |
| | 20 | μg/mL | E | + | + | O | O | O | O | O | O | O | O | O | O |
| | 10 | μg/mL | F | + | + | O | O | O | O | O | O | O | O | O | O |
| | 5 | μg/mL | G | + | + | O | O | O | O | O | O | O | O | O | O |

| | | | | | | | | | | | | | |
|---|---|---|---|---|---|---|---|---|---|---|---|---|---|
| No FluMAbs | H | + | + | + | + | + | + | + | + | + | + | O | O |

+: CPE
O: No CPE

TABLE 8
Experiment 4 - influenza virus A/H1N1 and FluMAbs CR6323
Schematic overview MW96: HA

| | | | | Undiluted | | | | | | | | | | Non- | |
|---|---|---|---|---|---|---|---|---|---|---|---|---|---|---|---|
| | | | | sample | | 1 | | 0.1 | | 0.01 | | 0.001 | | infected | |
| MOI | | | | 1 | 2 | 3 | 4 | 5 | 6 | 7 | 8 | 9 | 10 | 11 | 12 |
| Conc. | 320 | μg/mL | A | + | + | O | O | O | O | O | O | O | O | O | O |
| FluMabs | 160 | μg/mL | B | + | O | O | O | O | O | O | O | O | O | O | O |
| | 80 | μg/mL | C | O | O | O | O | O | O | O | O | O | O | O | O |
| | 40 | μg/mL | D | O | O | O | O | O | O | O | O | O | O | O | O |
| | 20 | μg/mL | E | O | O | O | O | O | O | O | O | O | O | O | O |
| | 10 | μg/mL | F | O | O | O | O | O | O | O | O | O | O | O | O |
| | 5 | μg/mL | G | O | O | O | O | O | O | O | O | O | O | O | O |

| | | | | | | | | | | | | | |
|---|---|---|---|---|---|---|---|---|---|---|---|---|---|
| No FluMAbs | H | + | + | + | + | + | + | + | + | + | + | O | O |

+: HA
O: No HA

TABLE 9
Experiment 5 - influenza B virus and FluMAbs CR11039 (CPE)
Schematic overview MW96 plate 1: CPE

| | | | | Undiluted = | | | | | | | | | | | |
|---|---|---|---|---|---|---|---|---|---|---|---|---|---|---|---|
| | | | | 1.7 × 10⁶ | | 8.5 × 10⁵ | | 4.2 × 10⁵ | | 2.1 × 10⁵ | | 1.1 × 10⁵ | | Non- | |
| Virus + FluMAbs | | | | TCID₅₀ | | TCID₅₀ | | TCID₅₀ | | TCID₅₀ | | TCID₅₀ | | infected | |
| Total volume: 100 μL | | | | 1 | 2 | 3 | 4 | 5 | 6 | 7 | 8 | 9 | 10 | 11 | 12 |
| Conc. | 12.8 | μg/mL | A | O | O | O | O | O | O | O | O | O | O | O | O |
| FluMAbs | 6.4 | μg/mL | B | O | O | O | O | O | O | O | O | O | O | O | O |
| | 3.2 | μg/mL | C | O | O | O | O | O | O | O | O | O | O | O | O |
| | 1.6 | μg/mL | D | O | O | O | O | O | O | O | O | O | O | O | O |
| | 0.8 | μg/mL | E | O | O | O | O | O | O | O | O | O | O | O | O |
| | 0.4 | μg/mL | F | O | O | O | O | O | O | O | O | O | O | O | O |
| | 0.2 | μg/mL | G | + | + | + | + | O | O | O | O | O | O | O | O |

| | | | | | | | | | | | | | |
|---|---|---|---|---|---|---|---|---|---|---|---|---|---|
| No FluMAbs | H | + | + | + | + | + | + | + | + | + | + | O | O |

+: CPE
O: No CPE

TABLE 10
Experiment 5 - influenza B virus and FluMAbs CR11039 (CPE)
Schematic overview MW96 plate 2: CPE

| | | | | 1.0 × 10⁴ | | 1.0 × 10³ | | 1.0 × 10² | | 1.0 × 10¹ | | 1.0 × 10⁰ | | Non- | |
|---|---|---|---|---|---|---|---|---|---|---|---|---|---|---|---|
| Virus + FluMAbs | | | | TCID₅₀ | | TCID₅₀ | | TCID₅₀ | | TCID₅₀ | | TCID₅₀ | | infected | |
| Total volume: 100 μL | | | | 1 | 2 | 3 | 4 | 5 | 6 | 7 | 8 | 9 | 10 | 11 | 12 |
| Conc. | 12.8 | μg/mL | A | O | O | O | O | O | O | O | O | O | O | O | O |
| FluMAbs | 6.4 | μg/mL | B | O | O | O | O | O | O | O | O | O | O | O | O |
| | 3.2 | μg/mL | C | O | O | O | O | O | O | O | O | O | O | O | O |
| | 1.6 | μg/mL | D | O | O | O | O | O | O | O | O | O | O | O | O |
| | 0.8 | μg/mL | E | O | O | O | O | O | O | O | O | O | O | O | O |
| | 0.4 | μg/mL | F | O | O | O | O | O | O | O | O | O | O | O | O |
| | 0.2 | μg/mL | G | O | O | O | O | O | O | O | O | O | O | O | O |

| | | | | | | | | | | | | | |
|---|---|---|---|---|---|---|---|---|---|---|---|---|---|
| No FluMAbs | H | + | + | + | + | + | + | + | + | + | + | O | O |

+: CPE
O: No CPE

TABLE 11
Experiment 5 - influenza B virus and FluMAbs CR11039 (HA)
Schematic overview MW96 plate 1: HA

| | | | | Undiluted = | | | | | | | | | | | |
|---|---|---|---|---|---|---|---|---|---|---|---|---|---|---|---|
| | | | | 1.7 × 10⁶ | | 8.5 × 10⁵ | | 4.2 × 10⁵ | | 2.1 × 10⁵ | | 1.1 × 10⁵ | | Non- | |
| Virus + FluMAbs | | | | TCID₅₀ | | TCID₅₀ | | TCID₅₀ | | TCID₅₀ | | TCID₅₀ | | infected | |
| Total volume: 100 μL | | | | 1 | 2 | 3 | 4 | 5 | 6 | 7 | 8 | 9 | 10 | 11 | 12 |
| Conc. | 12.8 | μg/mL | A | O | O | O | O | O | O | O | O | O | O | O | O |
| FluMAbs | 6.4 | μg/mL | B | O | O | O | O | O | O | O | O | O | O | O | O |
| | 3.2 | μg/mL | C | O | O | O | O | O | O | O | O | O | O | O | O |
| | 1.6 | μg/mL | D | O | O | O | O | O | O | O | O | O | O | O | O |
| | 0.8 | μg/mL | E | O | O | O | O | O | O | O | O | O | O | O | O |
| | 0.4 | μg/mL | F | + | + | O | O | O | O | O | O | O | O | O | O |
| | 0.2 | μg/mL | G | + | + | + | + | O | O | O | O | O | O | O | O |

| | | | | | | | | | | | | | |
|---|---|---|---|---|---|---|---|---|---|---|---|---|---|
| No FluMAbs | H | + | + | + | + | + | + | + | + | + | + | O | O |

+: HA
O: No HA

TABLE 12
Experiment 5 - influenza B virus and FluMAbs CR11039 (HA)
Schematic overview MW96 plate 2: HA

| | | | | 1.0 × 10⁴ | | 1.0 × 10³ | | 1.0 × 10² | | 1.0 × 10¹ | | 1.0 × 10⁰ | | Non- | |
|---|---|---|---|---|---|---|---|---|---|---|---|---|---|---|---|
| Virus + FluMAbs | | | | TCID₅₀ | | TCID₅₀ | | TCID₅₀ | | TCID₅₀ | | TCID₅₀ | | infected | |
| Total volume: 100 μL | | | | 1 | 2 | 3 | 4 | 5 | 6 | 7 | 8 | 9 | 10 | 11 | 12 |
| Conc. | 12.8 | μg/mL | A | O | O | O | O | O | O | O | O | O | O | O | O |
| FluMAbs | 6.4 | μg/mL | B | O | O | O | O | O | O | O | O | O | O | O | O |
| | 3.2 | μg/mL | C | O | O | O | O | O | O | O | O | O | O | O | O |
| | 1.6 | μg/mL | D | O | O | O | O | O | O | O | O | O | O | O | O |
| | 0.8 | μg/mL | E | O | O | O | O | O | O | O | O | O | O | O | O |
| | 0.4 | μg/mL | F | O | O | O | O | O | O | O | O | O | O | O | O |
| | 0.2 | μg/mL | G | O | O | O | O | O | O | O | O | O | O | O | O |

| | | | | | | | | | | | | | |
|---|---|---|---|---|---|---|---|---|---|---|---|---|---|
| No FluMAbs | H | + | + | + | + | + | + | + | + | + | + | O | O |

+: HA
O: No HA

TABLE 13
Experiment 6 - influenza B virus and FluMAbs CR11039
Schematic overview MW96 plate 1: CPE

| | | | | Undiluted = | | | | | | | | | | | |
|---|---|---|---|---|---|---|---|---|---|---|---|---|---|---|---|
| | | | | 1.7 × 10⁶ | | 8.5 × 10⁵ | | 4.2 × 10⁵ | | 2.1 × 10⁵ | | 1.1 × 10⁵ | | Non- | |
| Virus + FluMAbs | | | | TCID₅₀ | | TCID₅₀ | | TCID₅₀ | | TCID₅₀ | | TCID₅₀ | | infected | |
| Total volume: 100 μL | | | | 1 | 2 | 3 | 4 | 5 | 6 | 7 | 8 | 9 | 10 | 11 | 12 |
| Conc. | 12.8 | μg/mL | A | O | O | O | O | O | O | O | O | O | O | O | O |
| FluMAbs | 3.2 | μg/mL | B | O | O | O | O | O | O | O | O | O | O | O | O |
| | 0.8 | μg/mL | C | O | O | O | O | O | O | O | O | O | O | O | O |
| | 0.2 | μg/mL | D | + | + | + | + | O | O | O | O | O | O | O | O |
| | 0.05 | μg/mL | E | + | + | + | + | + | + | + | + | + | + | O | O |
| | 0.01 | μg/mL | F | ND | ND | + | + | + | + | + | + | + | + | O | O |
| | 3.1 | ng/mL | G | ND | ND | + | + | + | + | + | + | + | + | O | O |

| | | | | | | | | | | | | | |
|---|---|---|---|---|---|---|---|---|---|---|---|---|---|
| No FluMAbs | H | + | + | + | + | + | + | + | + | + | + | + | O |

+: CPE
O: No CPE
ND: Not done

TABLE 14
Experiment 6 - influenza B virus and FluMAbs CR11039
Schematic overview MW96 plate 1: HA

| | | | | Undiluted = | | | | | | | | | | | |
|---|---|---|---|---|---|---|---|---|---|---|---|---|---|---|---|
| | | | | 1.7 × 10⁶ | | 8.5 × 10⁵ | | 4.2 × 10⁵ | | 2.1 × 10⁵ | | 1.1 × 10⁵ | | Non- | |
| Virus + FluMAbs | | | | TCID₅₀ | | TCID₅₀ | | TCID₅₀ | | TCID₅₀ | | TCID₅₀ | | infected | |
| Total volume: 100 μL | | | | 1 | 2 | 3 | 4 | 5 | 6 | 7 | 8 | 9 | 10 | 11 | 12 |
| Conc. | 12.8 | μg/mL | A | O | O | O | O | O | O | O | O | O | O | O | O |
| FluMAbs | 3.2 | μg/mL | B | O | O | O | O | O | O | O | O | O | O | O | O |
| | 0.8 | μg/mL | C | O | O | O | O | O | O | O | O | O | O | O | O |
| | 0.2 | μg/mL | D | + | + | + | + | O | + | O | O | O | O | O | O |
| | 0.05 | μg/mL | E | + | + | + | + | + | + | + | + | + | + | O | O |
| | 0.01 | μg/mL | F | ND | ND | + | + | + | + | + | + | + | + | O | O |
| | 3.1 | ng/mL | G | ND | ND | + | + | + | + | + | + | + | + | O | O |

| | | | | | | | | | | | | | |
|---|---|---|---|---|---|---|---|---|---|---|---|---|---|
| No FluMAbs | H | + | + | + | + | + | + | + | + | + | + | + | O |

+: HA
O: No HA
ND: Not done

TABLE 15
Experiment 7 - influenza virus A/H1N1 and FluMAbs CR6323
Schematic overview MW96 plate 1: CPE

| | | | | 1.37 × 10⁵ | | 6.87 × 10⁴ | | 3.43 × 10⁴ | | 1.72 × 10⁴ | | 8.59 × 10³ | | Non- | |
|---|---|---|---|---|---|---|---|---|---|---|---|---|---|---|---|
| Virus + FluMAbs | | | | TCID₅₀ | | TCID₅₀ | | TCID₅₀ | | TCID₅₀ | | TCID₅₀ | | infected | |
| Total volume: 100 μL | | | | 1 | 2 | 3 | 4 | 5 | 6 | 7 | 8 | 9 | 10 | 11 | 12 |
| Conc. | 320 | μg/mL | A | O | O | O | O | O | O | O | O | O | O | O | O |
| FluMabs | 160 | μg/mL | B | O | O | O | O | O | O | O | O | O | O | O | O |
| | 80 | μg/mL | C | O | O | O | O | O | O | O | O | O | O | O | O |
| | 40 | μg/mL | D | O | O | O | O | O | O | O | O | O | O | O | O |
| | 20 | μg/mL | E | O | O | O | O | O | O | O | O | O | O | O | O |
| | 10 | μg/mL | F | O | O | O | O | O | O | O | O | O | O | O | O |
| | 5 | μg/mL | G | O | ND | O | ND | O | ND | O | ND | O | ND | O | ND |

| | | | | | | | | | | | | | |
|---|---|---|---|---|---|---|---|---|---|---|---|---|---|
| No FluMAbs | H | + | + | + | + | + | + | + | + | + | + | O | O |

+: CPE
O: No CPE
ND: Not done

TABLE 16
Experiment 7 - influenza virus A/H1N1 and FluMAbs CR6323
Schematic overview MW96 plate 1: HA

| | | | | 1.37 × 10⁵ | | 6.87 × 10⁴ | | 3.43 × 10⁴ | | 1.72 × 10⁴ | | 8.59 × 10³ | | Non- | |
|---|---|---|---|---|---|---|---|---|---|---|---|---|---|---|---|
| Virus + FluMAbs | | | | TCID₅₀ | | TCID₅₀ | | TCID₅₀ | | TCID₅₀ | | TCID₅₀ | | infected | |
| Total volume: 100 μL | | | | 1 | 2 | 3 | 4 | 5 | 6 | 7 | 8 | 9 | 10 | 11 | 12 |
| Conc. | 320 | μg/mL | A | + | + | O | O | O | O | O | O | O | O | O | O |
| FluMabs | 160 | μg/mL | B | + | + | O | O | O | O | O | O | O | O | O | O |
| | 80 | μg/mL | C | + | + | O | O | O | O | O | O | O | O | O | O |
| | 40 | μg/mL | D | + | + | O | O | O | O | O | O | O | O | O | O |
| | 20 | μg/mL | E | + | + | O | O | O | O | O | O | O | O | O | O |
| | 10 | μg/mL | F | + | + | + | + | O | O | O | O | O | O | O | O |
| | 5 | μg/mL | G | + | ND | + | ND | + | ND | + | ND | O | ND | O | ND |

| | | | | | | | | | | | | | |
|---|---|---|---|---|---|---|---|---|---|---|---|---|---|
| No FluMAbs | H | + | + | + | + | + | + | + | + | + | + | O | O |

+: HA
O: No HA
ND: Not done

TABLE 17
Experiment 8 - influenza virus A/H3N2 and FluMAbs CR8043 (CPE)
Schematic overview MW96 plate 1: CPE

| | | | | Undiluted = | | | | | | | | | | | |
|---|---|---|---|---|---|---|---|---|---|---|---|---|---|---|---|
| | | | | 4.05 × 10⁴ | | 2.03 × 10⁴ | | 1.01 × 10⁴ | | 5.07 × 10³ | | 2.53 × 10³ | | Non- | |
| Virus + FluMAbs | | | | TCID₅₀ | | TCID₅₀ | | TCID₅₀ | | TCID₅₀ | | TCID₅₀ | | infected | |
| Total volume: 100 μL | | | | 1 | 2 | 3 | 4 | 5 | 6 | 7 | 8 | 9 | 10 | 11 | 12 |
| Conc. | 640 | μg/mL | A | + | + | O | O | O | O | O | O | O | O | O | O |
| FluMabs | 320 | μg/mL | B | + | + | O | O | O | O | O | O | O | O | O | O |
| | 160 | μg/mL | C | + | + | O | O | O | O | O | O | O | O | O | O |
| | 80 | μg/mL | D | + | + | O | O | O | O | O | O | O | O | O | O |
| | 40 | μg/mL | E | + | + | + | + | O | + | O | O | O | O | O | O |
| | 20 | μg/mL | F | + | + | + | + | O | + | O | O | O | O | O | O |
| | 10 | μg/mL | G | + | + | + | + | + | + | + | + | O/+ | O/+ | O | O |

| | | | | | | | | | | | | | |
|---|---|---|---|---|---|---|---|---|---|---|---|---|---|
| No FluMAbs | H | + | ND | + | + | + | + | + | + | + | + | + | O |

+: CPE
O: No CPE
O/+: No consensus between scores
ND: Not done

TABLE 18
Experiment 8 - influenza virus A/H3N2 and FluMAbs CR8043 (CPE)
Schematic overview MW96 plate 2: CPE

| | | | | 1.27 × 10³ | | 6.34 × 10² | | 3.17 × 10² | | 1.58 × 10² | | 0.79 × 10² | | Non- | |
|---|---|---|---|---|---|---|---|---|---|---|---|---|---|---|---|
| Virus + FluMAbs | | | | TCID₅₀ | | TCID₅₀ | | TCID₅₀ | | TCID₅₀ | | TCID₅₀ | | infected | |
| Total volume: 100 μL | | | | 1 | 2 | 3 | 4 | 5 | 6 | 7 | 8 | 9 | 10 | 11 | 12 |
| Conc. | 640 | μg/mL | A | O | O | O/+ | + | O | O | O | O | O | O | O | O |
| FluMabs | 320 | μg/mL | B | O | O | O | O | O | O | O | O | O | O | O | O |
| | 160 | μg/mL | C | O | O | O | O | O | O/+ | O | + | O | O | O | O |
| | 80 | μg/mL | D | O | O | O | O | O | O | O | O | O | O | O | O |
| | 40 | μg/mL | E | O | O | O | O | O/+ | O | O | O | O | O | O | O |
| | 20 | μg/mL | F | O | O | O | O | O | O | + | + | O | O | O | O |
| | 10 | μg/mL | G | + | O | O | O | O | O | O | O | O | O | O | O |

| | | | | | | | | | | | | | |
|---|---|---|---|---|---|---|---|---|---|---|---|---|---|
| No FluMAbs | H | + | + | + | + | + | + | + | + | + | + | O | O |

+: CPE
O: No CPE
O/+: No consensus between scores
ND: Not done

TABLE 19
Experiment 8 - influenza virus A/H3N2 and FluMAbs CR8043 (HA)
Schematic overview MW96 plate 1: HA

| | | | | Undiluted = | | | | | | | | | | | |
|---|---|---|---|---|---|---|---|---|---|---|---|---|---|---|---|
| | | | | 4.05 × 10⁴ | | 2.03 × 10⁴ | | 1.01 × 10⁴ | | 5.07 × 10³ | | 2.53 × 10³ | | Non- | |
| Virus + FluMAbs | | | | TCID₅₀ | | TCID₅₀ | | TCID₅₀ | | TCID₅₀ | | TCID₅₀ | | infected | |
| Total volume: 100 μL | | | | 1 | 2 | 3 | 4 | 5 | 6 | 7 | 8 | 9 | 10 | 11 | 12 |
| Conc. | 640 | μg/mL | A | + | + | + | + | + | + | + | + | + | + | O | O |
| FluMabs | 320 | μg/mL | B | + | + | + | + | + | + | + | + | + | + | O | O |
| | 160 | μg/mL | C | + | + | + | + | + | + | + | + | + | + | O | O |
| | 80 | μg/mL | D | + | + | + | + | + | + | + | + | + | + | O | O |
| | 40 | μg/mL | E | + | + | + | + | + | + | + | + | + | + | O | O |
| | 20 | μg/mL | F | + | + | + | + | + | + | + | + | + | + | O | O |
| | 10 | μg/mL | G | + | + | + | + | + | + | + | + | + | + | O | O |

| | | | | | | | | | | | | | |
|---|---|---|---|---|---|---|---|---|---|---|---|---|---|
| No FluMAbs | H | + | ND | + | + | + | + | + | + | + | + | + | O |

+: HA
O: No HA
ND: Not done

TABLE 20
Experiment 8 - influenza virus A/H3N2 and FluMAbs CR8043 (HA)
Schematic overview MW96 plate 2: HA

| | | | | 1.27 × 10³ | | 6.34 × 10² | | 3.17 × 10² | | 1.58 × 10² | | 0.79 × 10² | | Non- | |
|---|---|---|---|---|---|---|---|---|---|---|---|---|---|---|---|
| Virus + FluMAbs | | | | TCID₅₀ | | TCID₅₀ | | TCID₅₀ | | TCID₅₀ | | TCID₅₀ | | infected | |
| Total volume: 100 μL | | | | 1 | 2 | 3 | 4 | 5 | 6 | 7 | 8 | 9 | 10 | 11 | 12 |
| Conc. | 640 | μg/mL | A | O | + | + | + | O | O | O | O | O | O | O | O |
| FluMabs | 320 | μg/mL | B | O | O | O | O | O | O | O | O | O | O | O | O |
| | 160 | μg/mL | C | O | + | O | O | O | O | O | + | O | O | O | O |
| | 80 | μg/mL | D | + | + | + | O | O | O | O | O | O | O | O | O |
| | 40 | μg/mL | E | O | + | + | + | + | O | O | O | O | O | O | O |
| | 20 | μg/mL | F | + | + | + | + | + | O | + | + | O | O | O | O |
| | 10 | μg/mL | G | + | + | + | + | + | O | O | O | O | O | O | O |

| | | | | | | | | | | | | | |
|---|---|---|---|---|---|---|---|---|---|---|---|---|---|
| No FluMAbs | H | + | + | + | + | + | + | + | + | + | + | O | O |

+: HA
O: No HA
ND: Not done

Based on the above, the following neutralizing capacity in MDCK cell culture was determined:

FluMAb CR8043:

Experiment 1: >5 μg/mL neutralizes 2-fold diluted A/H3N2 based on CPE.
>5 μg/mL neutralizes 200-fold diluted A/H3N2 based on HA.

Experiment 2: >10 μg/mL neutralizes 2-fold diluted A/H3N2 based on CPE.
>10 μg/mL neutralizes 200-fold diluted A/H3N2 based on HA.

Experiment 3: >10 μg/mL neutralizes 2-fold diluted A/H3N2 based on CPE.
>160 μg/mL neutralizes 20-fold diluted A/H3N2 based on HA;
>20 μg/mL neutralizes 200-fold diluted A/H3N2 based on HA.

Experiment 8: >80 μg/mL neutralizes 2-fold diluted A/H3N2 based on CPE.
>80 μg/mL neutralizes 128-fold diluted A/H3N2 based on HA;
>10 μg/mL neutralizes 256-fold diluted A/H3N2 based on HA.

FluMAb CR6323:

Experiment 4: >5 μg/mL neutralizes 7-fold diluted A/H1N1 based on CPE.
>5 μg/mL neutralizes 7-fold diluted A/H1N1 based on HA.

Experiment 7: >5 μg/mL neutralizes undiluted A/H1N1 based on CPE.
>20 μg/mL neutralizes 2-fold diluted A/H1N1 based on HA.

FluMAb CR11039:

Experiment 5: >0.4 μg/mL neutralizes undiluted influenza B virus (CPE).
>0.8 μg/mL neutralizes undiluted influenza B virus (HA).

Experiment 6: >0.8 μg/mL neutralizes undiluted influenza B virus (CPE).
>0.8 μg/mL neutralizes undiluted influenza B virus (HA).

Taking into account the lowest dilution of influenza virus and concentration FluMAb at which no CPE nor HA activity was observed, the following neutralizing capacity in MDCK cell culture was determined:

CR8043: >20 μg/mL neutralizes 200-fold diluted A/H3N2.

CR6323: >20 μg/mL neutralizes 2-fold diluted A/H1N1.

CR11039: >0.8 μg/mL neutralizes undiluted influenza B virus.

### Materials

Viruses (2011/2012)

| Influenza virus | TCID₅₀/mL |
|---|---|
| A/Victoria/210/2009 NYMC X-187 | Log 6.21 TCID₅₀/mL |
| A/California/7/2009 NYMC X-181 | Log 6.74 TCID₅₀/mL |
| B/Brisbane/60/2008 BX-35 | Log 7.83 TCID₅₀/mL |

During this study, use was made of the unique universal influenza antibodies CR8043. CR6323, and CR11039. All antibodies are of subclass IgG1 with kappa light-chains. The first two bind a conserved region in the stem of the hemagglutinin, while the latter binds a conserved region in the head of the hemagglutinin. The Examples described herein show neutralizing capacity as determined in MDCK cell culture. These results have been confirmed in a pilot study and GMP-release test, using MRC-5, Vero and adherent PER.C6® cells.

TABLE 21
Amino acid sequences of antibody CDR regions

| | HCDR1 | HCDR2 | HCDR3 | LCDR | LCDR2 | LCDR3 |
|---|---|---|---|---|---|---|
| | (SEQ | (SEQ | (SEQ | (SEQ | (SEQ | (SEQ |
| Anti- | ID | ID | ID | ID | ID | ID |
| body | NO:) | NO:) | NO:) | NO:) | NO:) | NO:) |
| CR6323 | SYGIS | DIIGM | SSGYY | RASQS | GASSR | QQYGS |
| | (1) | FGSTN | PAYL | VSSSY | AT(5) | SPRT |
| | | YAQNF | PH(3) | LA(4) | | (6) |
| | | QG(2) | | | | |
| CR8043 | AYSMH | WINTA | GASWD | KSSQS | WSSTR | HQYYT |
| | (7) | IGNTQ | ARGWS | VFSSS | ES(11) | APWT |
| | | YSQKF | GY(9) | TNKNY | | (12) |
| | | QD(8) | | LA(10) | | |
| CR11039 | GYAFT | INTYK | ARDWA | QDISD | GAS | QQYGN |
| | GYG | FNT | GPFGN | Y(16) | (17) | LPPT |
| | (13) | (14) | AFDV | | | (18) |
| | | | (15) | | | |

SEQUENCES

CR6323: Nucleotide sequence heavy chain (SEQ ID NO: 19)
gaggtgcagc tggtggagtc tggggctgag gtgaagaagc cagggtcctc ggtgaaggtc 60
tcctgtaagg cctctggagg caccttctcc agctatggta tcagctgggt gcgacaggcc 120
cctggacaag ggcttggagt gatgggagac atcatcggta tgtttggttc aacaaactac 180
gcacagaact tccagggcag actcacgatt accgcggacg aatccacgag cacagcctac 240
atggagctga gcagcctgag atctgaggac acggccgtgt attactgtgc gagaagtagt 300
ggttattacc ctgcatacct cccccactgg ggccagggca ccttggtcac cgtctcgagt 360
gctagcacca agggccccag cgtgttcccc ctggccccca gcagcaagag caccagcggc 420
ggcacagccg ccctgggctg cctggtgaag gactacttcc ccgagcccgt gaccgtgagc 480
tggaacagcg gcgccttgac cagcggcgtg cacaccttcc ccgccgtgct gcagagcagc 540
ggcctgtaca gcctgagcag cgtggtgacc gtgcccagca gcagcctggg cacccagacc 600
tacatctgca acgtgaacca caagcccagc aacaccaagg tggacaaacg cgtggagccc 660
aagagctgcg acaagaccca cacctgcccc ccctgccctg cccccgagct gctgggcgga 720
ccctccgtgt tcctgttccc ccccaagccc aaggacaccc tcatgatcag ccggaccccc 780
gaggtgacct gcgtggtggt ggacgtgagc cacgaggacc ccgaggtgaa gttcaactgg 840
tacgtggacg gcgtggaggt gcacaacgcc aagaccaagc cccgggagga gcagtacaac 900
agcacctacc gggtggtgag cgtgctcacc gtgctgcacc aggactggct gaacggcaag 960
gagtacaagt gcaaggtgag caacaaggcc ctgcctgccc ccatcgagaa gaccatcagc 1020
aaggccaagg gccagccccg ggagccccag gtgtacaccc tgccccccag ccgggaggag 1080
atgaccaaga accaggtgtc cctcacctgt ctggtgaagg gcttctaccc cagcgacatc 1140
gccgtggagt gggagagcaa cggccagccc gagaacaact acaagaccac cccccctgtg 1200
ctggacagcg acggcagctt cttcctgtac agcaagctca ccgtggacaa gagccggtgg 1260
cagcagggca acgtgttcag ctgcagcgtg atgcacgagg ccctgcacaa ccactacacc 1320
cagaagagcc tgagcctgag ccccggcaag 1350
CR6323: Amino acid sequence heavy chain (SEQ ID NO: 20);_Vh amino acids 1-120
E V Q L V E S G A E V K K P G S S V K V S C K A S G G T F S S Y G I S W V R
Q A P G Q G L E W M G D I I G M F G S T N Y A Q N F Q G R L T I T A D E S T
S T A Y M E L S S L R S E D T A V Y Y C A R S S G Y Y P A Y L P H W G Q G T
L V T V S S A S T K G P S V F P L A P S S K S T S G G T A A L G C L V K D Y
F P E P V T V S W N S G A L T S G V H T F P A V L Q S S G L Y S L S S V V T
V P S S S L G T Q T Y I C N V N H K P S N T K V D K R V E P K S C D K T H T
C P P C P A P E L L G G P S V F L F P P K P K D T L M I S R T P E V T C V V
V D V S H E D P E V K F N W Y V D G V E V H N A K T K P R E E Q Y N S T Y R
V V S V L T V L H Q D W L N G K E Y K C K V S N K A L P A P I E K T I S K A
K G Q P R E P Q V Y T L P P S R E E M T K N Q V S L T C L V K G F Y P S D I
A V E W E S N G Q P E N N Y K T T P P V L D S D G S F F L Y S K L T V D K S
R W Q Q G N V F S C S V M H E A L H N H Y T Q K S L S L S P G K
CR6323: Nucleotide sequence of light chain (SEQ ID NO: 21)
gaaattgtgt tgacccagtc tccaggcacc ctgtctttgt ctccagggga aagagccacc 60
ctctcctgca gggccagtca gagtgttagc agcagctact tagcctggta ccagcagaaa 120
cctggccagg ctcccaggct cctcatctat ggtgcatcca gcagggccac tggcatccca 180
gacaggttca gtggcagtgg gtctgggaca gacttcactc tcaccatcag cagactggag 240
cctgaagatt ttgcagtgta ttactgtcag cagtatggta gctcacccag aactttcggc 300
ggagggacca aggtggagat caaacgtgcg gccgcaccca gcgtgttcat cttccccccc 360
tccgacgagc agctgaagag cggcaccgcc agcgtggtgt gcctgctgaa caacttctac 420
ccccgggagg ccaaggtgca gtggaaggtg gacaacgccc tgcagagcgg caacagccag 480
gagagcgtga ccgagcagga cagcaaggac tccacctaca gcctgagcag caccctcacc 540
ctgagcaagg ccgactacga gaagcacaag gtgtacgcct gcgaggtgac ccaccagggc 600
ctgagcagcc ccgtgaccaa gagcttcaac cggggcgagt gt 642
CR6323: Amino acid sequence light chain (SEQ ID NO: 22); VI amino acids 1-109
E I V L T Q S P G T L S L S P G E R A T L S C R A S Q S V S S S Y L A W Y Q Q K P G Q
A P R L L I Y G A S S R A T G I P D R F S G S G S G T D F T L T I S R L E P E D F A V Y
Y C Q Q Y G S S P R T F G G G T K V E I K R A A A P S V F I F P P S D E Q L K S G T A S
V V C L L N N F Y P R E A K V Q W K V D N A L Q S G N S Q E S V T E Q D S K D S T Y
S L S S T L T L S K A D Y E K H K V Y A C E V T H Q G L S S P V T K S F N R G E C
CR8043: Nucleotide sequence variable region heavy chain (SEQ ID NO: 23)
CAGGTGCAGCTGGTGCAGTCTGGGGCTGAGGTGAAGAAGCCTGGGGCCTCAGTGAA
GCTTTCCTGCAAGGCTTCTGGATACACCTTCACTGCCTATTCTATGCATTGGGTGCGC
CAGGCCCCCGGACAAAGCCTTGAGTGGTTGGGATGGATCAACACTGCCATCGGTAA
CACACAATATTCACAGAAGTTCCAGGACAGAGTCACCATTACCAGGGACACATCTG
CGCGCACATCGTACATGGAACTGAGCAGCCTGAGATCTGGAGACACGGCTGTCTATT
TCTGTGCGAGAGGGGCCTCTTGGGACGCCCGTGGGTGGTCTGGCTAC
CR8043: Amino acid sequence variable region heavy chain (SEQ ID NO: 24)
QVQLVQSGAEVKKPGASVKLSCKASGYTFTAYSMHWVRQAPGQSLEWLGWINTAIGN
TQYSQKFQDRVTITRDTSARTSYMELSSLRSGDTAVYFCARGASWDARGWSGY
CR8043: Nucleotide sequence variable region light chain (SEQ ID NO: 25)
GACATCCAGWTGACCCAGTCTCCAGACTCCCTGGCTGTGTCTCTGGGCGAGAGGGC
CACCATCAACTGCAAGTCCAGCCAGAGTGTTTTTTCCAGCTCCACCAATAAGAACTA
CTTAGCTTGGTACCAGCAGAAACCAGGACAGCCTCCTAAGGTGCTAATTTACTGGTC
ATCTACCCGGGAATCCGGGGTCCCTGACCGATTCAGTGCCAGCGGGTCTGGGACAG
ATTTCACTCTCACCATCAGCAGCCTGCAGGCTGCAGATGTGGCAGTTTATTACTGTC
ACCAATATTATACTGCTCCGTGGACG
CR8043: Amino acid sequuence variable region light chain (SEQ ID NO: 26)
DIQXTQSPDSLAVSLGERATINCKSSQSVFSSSTNKNYLAWYQQKPGQPPKVLIYWSSTR
ESGVPDRFSASGSGTDFTLTISSLQAADVAVYYCHQYYTAPWT
CR11039: Nucleotide sequence variable region heavy chain (SEQ ID NO: 27)
GAGGTCCAGCTGGTACAGTCTGGAGCAGAGGTGAAAAAGCCCGGGGAGTCTGTGAA
GATCTCCTGTAAGACTTCTGGTTACGCCTTTACCGGCTACGGTATCAGCTGGGTGCG
ACAGGCCCCTGGACAAGGCCTTGAGTGGATGGGATGGATCAACACTTACAAATTTA
ACACAAATTATGCACAGAACCTGCAGGGCAGAGTCACCATGACCATAGACACATCC
ACGAGCGCAGCCTACATGGAGCTGAGGAGCCTGAGATATGAGGACACGGCCGTATA
TTTCTGTGCGAGAGACTGGGCTGGGCCGTTTGGGAATGCTTTTGATGTCTGGGGCCA
GGGGACAATGGTCACCGTCTCGAGCG
CR11039: Amino acid sequence variable region heavy chain (SEQ ID NO: 28)
EVQLVQSGAEVKKPGESVKISCKTSGYAFTGYGISWVRQAPGQGLEWMGWINTYKFNT
NYAQNLQGRVTMTIDTSTSAAYMELRSLRYEDTAVYFCARDWAGPGNAFDVWGQGT
MVTVSS
CR11039: Nucleotide sequence variable region light chain (SEQ ID NO: 29)
ACATCCAGATGACCCAGTCTCCATCTTCCCTGTCTGCATCTATAGGAGACAGAGTCG
CCATCACTTGCCAGGCGAGTCAGGACATTAGCGACTATTTAAATTGGTATCAGCAAC
AACCAGGGAAAGCCCCTAAGCTCCTGGCTCTACGGTGCATCCAATTTGGAAACAGGG
GTCCCATCAAGGTTCAGTGGAAGTGGATCTGGGACAGATTTTACTTTCACCATCAGC
AGCCTGCAGCCTGAAGACATTGCAACATATTATTGTCAACAGTATGGTAATCTCCCT
CCGACTTTCGGCGGGGGGACCAAGCTGGAGATCAAAC
CR11039: Amino acide sequence variable region light chain (SEQ ID NO: 30)
IQMTQSPSSLSASIGDRVAITCQASQDISDYLNWYQQQPGKAPKLLLYGASNLETGVPSR
FSGSGSGTDFTFTISSLQPEDIATYYCQQYGNLPPTFGGGTKLEIK

1. A method for testing for the presence of extraneous agent(s) in a composition comprising influenza virus, the method comprising:
neutralizing the influenza virus in said composition utilizing one or more influenza virus-neutralizing monoclonal antibodies, and/or antigen-binding fragments thereof that neutralize influenza virus.

2. The method according to claim 1, wherein the one or more influenza virus-neutralizing antibodies are selected from the group consisting of:
(i) an antibody comprising a heavy chain CDR1 region comprising SEQ ID NO: 1, a heavy chain CDR2 region comprising SEQ ID NO: 2 and a heavy chain CDR3 region comprising SEQ ID NO: 3; and a light chain CDR1 region comprising SEQ ID NO:4, a light chain CDR2 region comprising SEQ ID NO: 5 and a light chain CDR3 region comprising SEQ ID NO: 6;
(ii) an antibody comprising a heavy chain CDR1 region comprising SEQ ID NO:7, a heavy chain CDR2 region comprising SEQ ID NO: 8 and a heavy chain CDR3 region comprising SEQ ID NO: 9; and a light chain CDR1 region comprising SEQ ID NO:10, a light chain CDR2 region comprising SEQ ID NO: 11 and a light chain CDR3 region comprising SEQ ID NO: 12; and
(iii) an antibody comprising a heavy chain CDR1 region comprising SEQ ID NO:13, a heavy chain CDR2 region comprising SEQ ID NO: 14 and a heavy chain CDR3 region comprising SEQ ID NO: 15; and a light chain CDR1 region comprising SEQ ID NO:16, a light chain CDR2 region comprising SEQ ID NO: 17 and a light chain CDR3 region comprising SEQ ID NO: 18.

3. The method according to claim 1, wherein the one or more influenza virus-neutralizing antibodies are selected from the group consisting of:
(i) an antibody comprising a heavy chain variable region comprising the amino acids 1-120 of SEQ ID NO: 20 and a light chain variable region comprising the amino acid residues 1-109 of SEQ ID NO: 22;
(ii) an antibody comprising a heavy chain variable region comprising the amino acid sequence of SEQ ID NO: 24 and a light chain variable region comprising the amino acid sequence of SEQ ID NO: 26; and
(iii) an antibody comprising a heavy chain variable region comprising the amino acid sequence of SEQ ID NO: 28 and a light chain variable region comprising the amino acid sequence of SEQ ID NO: 30.

4. The method according to claim 1, 2 or 3, wherein the influenza virus has been propagated in a culture of a mammalian cell line.

5. The method according to claim 4, wherein the mammalian cell line is a PER.C6® cell line, a MDCK cell line, or a Vero cell line.

6. The method according to claim 1, wherein the influenza virus is an influenza A virus of phylogenetic group 1.

7. The method according to claim 1, wherein the influenza virus is an influenza A virus of phylogenetic group 2.

8. The method according to claim 1, wherein the influenza virus is an influenza B virus.

9. A neutralizing agent for extraneous agent testing comprising:
an influenza virus-neutralizing monoclonal, antibody selected from the group consisting of:
(i) an antibody comprising:
a heavy chain CDR1 region comprising SEQ ID NO: 1, a heavy chain CDR2 region comprising SEQ ID NO: 2, and a heavy chain CDR3 region comprising SEQ ID NO: 3; and
a light chain CDR1 region comprising SEQ ID NO:4, a light chain CDR2 region comprising SEQ ID NO: 5, and a light chain CDR3 region comprising SEQ ID NO: 6;
(ii) an antibody comprising:
a heavy chain CDR1 region comprising SEQ ID NO:7, a heavy chain CDR2 region comprising SEQ ID NO: 8, and a heavy chain CDR3 region comprising SEQ ID NO: 9; and
a light chain CDR1 region comprising SEQ ID NO:10, a light chain CDR2 region comprising SEQ ID NO: 11 and a light chain CDR3 region comprising SEQ ID NO: 12;
(iii) an antibody comprising:
a heavy chain CDR1 region comprising SEQ ID NO:13, a heavy chain CDR2 region comprising SEQ ID NO: 14, and a heavy chain CDR3 region comprising SEQ ID NO: 15; and
a light chain CDR1 region comprising SEQ ID NO:16, a light chain CDR2 region comprising SEQ ID NO: 17, and a light chain CDR3 region comprising SEQ ID NO: 18,
(iv) a functional variant of any thereof, and
(v) an antigen-binding fragment of any thereof,
having activity as a neutralizing agent for extraneous agent testing.

10. A method for preparing an influenza vaccine, the method comprising:
testing an influenza virus composition for the presence of extraneous agents,
wherein the influenza virus in said influenza virus composition is neutralized prior to said testing utilizing one or more influenza virus-neutralizing monoclonal antibodies, and/or functional variants, and/or antigen-binding fragments thereof that neutralize influenza virus.

11. The method according to claim 10, wherein the one or more influenza virus-neutralizing antibodies are selected from the group consisting of:
(i) an antibody comprising a heavy chain CDR1 region comprising SEQ ID NO: 1, a heavy chain CDR2 region comprising SEQ ID NO: 2 and a heavy chain CDR3 region comprising SEQ ID NO: 3; and a light chain CDR1 region comprising SEQ ID NO:4, a light chain CDR2 region comprising SEQ ID NO: 5 and a light chain CDR3 region comprising SEQ ID NO: 6;
(ii) an antibody comprising a heavy chain CDR1 region comprising SEQ ID NO:7, a heavy chain CDR2 region comprising SEQ ID NO: 8 and a heavy chain CDR3 region comprising SEQ ID NO: 9; and a light chain CDR1 region comprising SEQ ID NO:10, a light chain CDR2 region comprising SEQ ID NO: 11 and a light chain CDR3 region comprising SEQ ID NO: 12; and
(iii) an antibody comprising a heavy chain CDR1 region comprising SEQ ID NO:13, a heavy chain CDR2 region comprising SEQ ID NO: 14 and a heavy chain CDR3 region comprising SEQ ID NO: 15; and a light chain CDR1 region comprising SEQ ID NO:16, a light chain CDR2 region comprising SEQ ID NO: 17 and a light chain CDR3 region comprising SEQ ID NO: 18.

12. The method according to claim 10, wherein the one or more influenza virus-neutralizing antibodies are selected from the group consisting of:
(i) an antibody comprising a heavy chain variable region comprising the amino acids 1-120 of SEQ ID NO: 20 and a light chain variable region comprising the amino acid residues 1-109 of SEQ ID NO: 22;
(ii) an antibody comprising a heavy chain variable region comprising the amino acid sequence of SEQ ID NO: 24 and a light chain variable region comprising the amino acid sequence of SEQ ID NO: 26; and
(iii) an antibody comprising a heavy chain variable region comprising the amino acid sequence of SEQ ID NO: 28 and a light chain variable region comprising the amino acid sequence of SEQ ID NO: 30.

13. The method according to claim 10, wherein the influenza vaccine is an inactivated virus vaccine.

14. The method according to claim 13, wherein the influenza vaccine is a whole virus vaccine, a split virus vaccine, a viral subunit vaccine, or a virosomal vaccine.

15. The method according to claim 10, wherein the vaccine is a trivalent or a quadrivalent influenza vaccine.

16. The method according to claim 6, wherein the influenza A virus of phylogenetic group 1 comprises HA of the H1, H2, H5 or 1-19 subtype.

17. The method according to claim 7, wherein the influenza A virus of phylogenetic group 2 comprises HA of the H3, H7, or H10 subtype.

18. The method according to claim 8, wherein the influenza B virus is an influenza B virus strain from the B/Yamagata or B/Victoria lineage.


SEQUENCE LISTING (30 sequences)

SEQ ID NO: 1 - protein, 5 aa, Artificial Sequence
  CR6323 HCDR1
SYGIS

SEQ ID NO: 2 - protein, 17 aa, Artificial Sequence
  CR6323 HCDR2
DIIGMFGSTNYAQNFQG

SEQ ID NO: 3 - protein, 11 aa, Artificial Sequence
  CR6323 HCDR3
SSGYYPAYLPH

SEQ ID NO: 4 - protein, 12 aa, Artificial Sequence
  CR6323 LCDR1
RASQSVSSSYLA

SEQ ID NO: 5 - protein, 7 aa, Artificial Sequence
  CR6323 LCDR2
GASSRAT

SEQ ID NO: 6 - protein, 9 aa, Artificial Sequence
  CR6323 LCDR3
QQYGSSPRT

SEQ ID NO: 7 - protein, 5 aa, Artificial Sequence
  CR8043 HCDR1
AYSMH

SEQ ID NO: 8 - protein, 17 aa, Artificial Sequence
  CR8043 HCDR2
WINTAIGNTQYSQKFQD

SEQ ID NO: 9 - protein, 12 aa, Artificial Sequence
  CR8043 HCDR3
GASWDARGWSGY

SEQ ID NO: 10 - protein, 17 aa, Artificial Sequence
  CR8043 LCDR1
KSSQSVFSSSTNKNYLA

SEQ ID NO: 11 - protein, 7 aa, Artificial Sequence
  CR8043 LCDR2
WSSTRES

SEQ ID NO: 12 - protein, 9 aa, Artificial Sequence
  CR8043 LCDR3
HQYYTAPWT

SEQ ID NO: 13 - protein, 8 aa, Artificial Sequence
  CR11039 HCDR1
GYAFTGYG

SEQ ID NO: 14 - protein, 8 aa, Artificial Sequence
  CR11039 HCDR2
INTYKFNT

SEQ ID NO: 15 - protein, 14 aa, Artificial Sequence
  CR11039 HCDR3
ARDWAGPFGNAFDV

SEQ ID NO: 16 - protein, 6 aa, Artificial Sequence
  CR11039 LCDR1
QDISDY

SEQ ID NO: 17 - protein, 3 aa, Artificial Sequence
  CR11039 LCDR2
GAS

SEQ ID NO: 18 - protein, 9 aa, Artificial Sequence
  CR11039 LCDR3
QQYGNLPPT

SEQ ID NO: 19 - DNA, 1350 bp, Artificial Sequence
  CR6323 Nucleotide sequence heavy chain
gaggtgcagctggtggagtctggggctgaggtgaagaagccagggtcctcggtgaaggtctcctgtaaggcctctggaggcaccttctccagctatggtatcagctgggtgcgacaggcccctggacaagggcttgagtggatgggagacatcatcggtatgtttggttcaacaaactacgcacagaacttccagggcagactcacgattaccgcggacgaatccacgagcacagcctacatggagctgagcagcctgagatctgaggacacggccgtgtattactgtgcgagaagtagtggttattaccctgcatacctcccccactggggccagggcaccttggtcaccgtctcgagtgctagcaccaagggccccagcgtgttccccctggcccccagcagcaagagcaccagcggcggcacagccgccctgggctgcctggtgaaggactacttccccgagcccgtgaccgtgagctggaacagcggcgccttgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaaacgcgtggagcccaagagctgcgacaagacccacacctgccccccctgccctgcccccgagctgctgggcggaccctccgtgttcctgttcccccccaagcccaaggacaccctcatgatcagccggacccccgaggtgacctgcgtggtggtggacgtgagccacgaggaccccgaggtgaagttcaactggtacgtggacggcgtggaggtgcacaacgccaagaccaagccccgggaggagcagtacaacagcacctaccgggtggtgagcgtgctcaccgtgctgcaccaggactggctgaacggcaaggagtacaagtgcaaggtgagcaacaaggccctgcctgcccccatcgagaagaccatcagcaaggccaagggccagccccgggagccccaggtgtacaccctgccccccagccgggaggagatgaccaagaaccaggtgtccctcacctgtctggtgaagggcttctaccccagcgacatcgccgtggagtgggagagcaacggccagcccgagaacaactacaagaccaccccccctgtgctggacagcgacggcagcttcttcctgtacagcaagctcaccgtggacaagagccggtggcagcagggcaacgtgttcagctgcagcgtgatgcacgaggccctgcacaaccactacacccagaagagcctgagcctgagccccggcaag

SEQ ID NO: 20 - protein, 450 aa, Artificial Sequence
  CR6323 Amino acid sequence heavy chain
EVQLVESGAEVKKPGSSVKVSCKASGGTFSSYGISWVRQAPGQGLEWMGDIIGMFGSTNYAQNFQGRLTITADESTSTAYMELSSLRSEDTAVYYCARSSGYYPAYLPHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 642 bp, Artificial Sequence
  CR6323 Nucleotide sequence of light chain
gaaattgtgttgacccagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacccagaactttcggcggagggaccaaggtggagatcaaacgtgcggccgcacccagcgtgttcatcttccccccctccgacgagcagctgaagagcggcaccgccagcgtggtgtgcctgctgaacaacttctacccccgggaggccaaggtgcagtggaaggtggacaacgccctgcagagcggcaacagccaggagagcgtgaccgagcaggacagcaaggactccacctacagcctgagcagcaccctcaccctgagcaaggccgactacgagaagcacaaggtgtacgcctgcgaggtgacccaccagggcctgagcagccccgtgaccaagagcttcaaccggggcgagtgt

SEQ ID NO: 22 - protein, 214 aa, Artificial Sequence
  CR6323 Amino acid sequence light chain
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPRTFGGGTKVEIKRAAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - DNA, 330 bp, Artificial Sequence
  CR8043  Nucleotide sequence variable region heavy chain
caggtgcagctggtgcagtctggggctgaggtgaagaagcctggggcctcagtgaagctttcctgcaaggcttctggatacaccttcactgcctattctatgcattgggtgcgccaggcccccggacaaagccttgagtggttgggatggatcaacactgccatcggtaacacacaatattcacagaagttccaggacagagtcaccattaccagggacacatctgcgcgcacatcgtacatggaactgagcagcctgagatctggagacacggctgtctatttctgtgcgagaggggcctcttgggacgcccgtgggtggtctggctac

SEQ ID NO: 24 - protein, 110 aa, Artificial Sequence
  CR8043 Amino acid sequence variable region heavy chain
QVQLVQSGAEVKKPGASVKLSCKASGYTFTAYSMHWVRQAPGQSLEWLGWINTAIGNTQYSQKFQDRVTITRDTSARTSYMELSSLRSGDTAVYFCARGASWDARGWSGY

SEQ ID NO: 25 - DNA, 309 bp, Artificial Sequence
  CR8043 Nucleotide sequence variable region light chain (
gacatccagwtgacccagtctccagactccctggctgtgtctctgggcgagagggccaccatcaactgcaagtccagccagagtgttttttccagctccaccaataagaactacttagcttggtaccagcagaaaccaggacagcctcctaaggtgctaatttactggtcatctacccgggaatccggggtccctgaccgattcagtgccagcgggtctgggacagatttcactctcaccatcagcagcctgcaggctgcagatgtggcagtttattactgtcaccaatattatactgctccgtggacg

SEQ ID NO: 26 - protein, 103 aa, Artificial Sequence
  CR8043 Amino acid sequence variable region light chain
DIQXTQSPDSLAVSLGERATINCKSSQSVFSSSTNKNYLAWYQQKPGQPPKVLIYWSSTRESGVPDRFSASGSGTDFTLTISSLQAADVAVYYCHQYYTAPWT

SEQ ID NO: 27 - DNA, 364 bp, Artificial Sequence
  CR11039  Nucleotide sequence variable region heavy chain
gaggtccagctggtacagtctggagcagaggtgaaaaagcccggggagtctgtgaagatctcctgtaagacttctggttacgcctttaccggctacggtatcagctgggtgcgacaggcccctggacaaggccttgagtggatgggatggatcaacacttacaaatttaacacaaattatgcacagaacctgcagggcagagtcaccatgaccatagacacatccacgagcgcagcctacatggagctgaggagcctgagatatgaggacacggccgtatatttctgtgcgagagactgggctgggccgtttgggaatgcttttgatgtctggggccaggggacaatggtcaccgtctcgagcg

SEQ ID NO: 28 - protein, 121 aa, Artificial Sequence
  CR11039  Amino acid sequence variable region heavy chain
EVQLVQSGAEVKKPGESVKISCKTSGYAFTGYGISWVRQAPGQGLEWMGWINTYKFNTNYAQNLQGRVTMTIDTSTSAAYMELRSLRYEDTAVYFCARDWAGPFGNAFDVWGQGTMVTVSS

SEQ ID NO: 29 - DNA, 321 bp, Artificial Sequence
  CR11039  Nucleotide sequence variable region light chain
acatccagatgacccagtctccatcttccctgtctgcatctataggagacagagtcgccatcacttgccaggcgagtcaggacattagcgactatttaaattggtatcagcaacaaccagggaaagcccctaagctcctgctctacggtgcatccaatttggaaacaggggtcccatcaaggttcagtggaagtggatctgggacagattttactttcaccatcagcagcctgcagcctgaagacattgcaacatattattgtcaacagtatggtaatctccctccgactttcggcggggggaccaagctggagatcaaac

SEQ ID NO: 30 - protein, 106 aa, Artificial Sequence
  CR11039  Amino acid sequence variable region light chain
IQMTQSPSSLSASIGDRVAITCQASQDISDYLNWYQQQPGKAPKLLLYGASNLETGVPSRFSGSGSGTDFTFTISSLQPEDIATYYCQQYGNLPPTFGGGTKLEIK